(19) United States
(12) Patent Application Publication
(10) Pub. No.: US 2010/0069476 A1
(43) Pub. Date: Mar. 18, 2010

(54) COMPOSITIONS AND METHODS FOR REDUCTION OF CUTANEOUS PHOTOAGEING

(75) Inventors: Yukihiko HARA, Tokyo (JP); Santosh K. Katiyar, Vestavia Hills, AL (US)

Correspondence Address: FISH & ASSOCIATES, PC;ROBERT D. FISH, 2603 Main Street, Suite 1000, Irvine, CA, 92614-6232, US

(73) Assignee: MITSUI NORIN CO., LTD, Tokyo (JP)

(21) Appl. No.: 11/914,590

(22) PCT Filed: May 17, 2005
(86) PCT No.: PCT/US2005/017335

§ 371 Date: Aug. 20, 2009

(51) Int. Cl. A61K 31/353; A61P 17/18; A61K 31/375

(52) U.S. Cl. 514/456; 514/474

(57) ABSTRACT

Compositions and methods are provided in which a topical hydrophilic formulation includes a catechin and a hydrophilic antioxidant in a hydrophilic composition at a ratio of between 2.3 to 1.7 (by weight), ad wherein the catechin and the antioxidant are present in an amount such that application of the composition to skin will deposit the catechin at a dosage of between 0.7 mg/cm2 and 1.3 mg/cm2. Especially preferred catechins include green tea catechins, and particularly EGCG, while preferred antioxidants include ascorbic acid and derivatives thereof.

### FIELD OF THE INVENTION

The field of the invention is compositions and methods for treatment and prevention of photoageing, and especially photoageing of skin.

### BACKGROUND OF THE INVENTION

Aging of skin may be characterized as a progressive loss of function and resiliency of the skin to numerous stress conditions, and is often manifested by increased susceptibility to injury and disease. Among other factors, conditions associated with aged skin are primarily attributable to a genetically determined degenerative process, and repeated extraneous insults, and particularly exposure to solar ultraviolet light (photoageing).

Photoageing accounts in many cases for wrinkling, mottled hyperpigmentation and/or depigmentation, coarsening, roughness, poor elastic recoil, and bruisability of the skin. Over time, lesions may develop and eventually lead to in situ skin cancers (e.g., actinic keratoses) or invasive skin cancers. Photoageing may be slowed down by avoidance of exposure to ultraviolet (UV) light, and/or by use of sunscreens that absorb selected portions of the UV spectrum. For example, numerous sunscreen agents are described in “Sunscreens: Regulations And Commercial Development” by Nadim Shaath (Marcel Dekker; 3rd Ed edition; ISBN: 0824757947). Additionally, numerous natural and synthetic compounds have been included into sunscreens. For example, green tea polyphenols or extracts (e.g., those including EGCG [epigallocatechin gallate]) have been added to sunscreens as agents to reduce inflammation and/or to provide antioxidant effect. However, to achieve significant effect, the concentration of such polyphenols or extracts must be relatively high. Moreover, and particularly in hydrophilic formulations, such polyphenols and extracts are relatively unstable and quickly degrade or polymerize. Other natural and synthetic compounds to reduce photoageing include vitamin E, collagen, hydrating agents, etc., which have at least some reported beneficial effects. However, most, if not all of the currently known additives to reduce photoageing provide only temporary protection, or have little or even no reproducible effect.

Thus, while numerous compositions and methods for reduction of photoageing are known in the art, all or almost all of them, suffer from one or more disadvantages. Therefore, there is still a need for improved compositions and methods for reduction of photoageing.

### SUMMARY OF THE INVENTION

The present invention is directed to compositions and methods comprising green tea polyphenols in combination with synergistic amounts of an antioxidant. Such compositions, when topically applied to skin (preferably prior to sun exposure), will reduce photoageing in the skin. Most preferably, the green tea polyphenols comprise polyphenon E, or one or more components thereof, the antioxidant is ascorbic acid or a derivative thereof, and the topical formulation is a hydrophilic topical formulation.

In one aspect of the inventive subject matter, a topical hydrophilic composition for reducing photoageing comprises a catechin (most preferably epigallocatechin gallate) and a hydrophilic antioxidant (most preferably ascorbic acid) at a ratio of between 2.3 to 1.7 (by weight) and present in an amount such that application of the composition will deposit the catechin at a dosage of between 0.7 mg/cm² and 1.3 mg/cm². In further preferred aspects of the inventive subject matter, the catechin is provided with a plurality of additional catechins (e.g., in form of polyphenon E), and the ascorbic acid may optionally be substituted. Where desired, contemplated topical formulations may also include a UV absorbing compound, and especially contemplated UV absorbing compounds will have a molar extinction coefficient of at least 1000 cm⁻¹ at a wavelength of between 290 nm to 390 nm.

In especially preferred topical compositions, the ratio between the catechin and the antioxidant is between 2.1 to 1.9. Most typically, the reduction of photoageing of skin using contemplated compositions is characterized by a reduction of UV-induced skin thickness, a reduction of hydrogen peroxide radical formation in skin, a reduction of protein oxidation in skin, and/or a reduction of expression of a matrix metalloproteinase. For example, a typical composition may provide a reduction of UV-induced skin thickness of 75% or even more as compared to non-treatment, and/or a reduction of hydrogen peroxide radical formation of at least 50% as compared to non-treatment.

Consequently, in another aspect of the inventive subject matter, a method of reducing photoageing includes a step in which a catechin and a hydrophilic antioxidant in a hydrophilic composition are provided at a ratio of between 2.3 to 1.7 (by weight). In another step, the composition is applied to the skin in an amount such that the catechin is present on the skin at a quantity of between 0.7 mg/cm² and 1.3 mg/cm². In still another step, the so treated skin is then irradiated.

Various objects, features, aspects and advantages of the present invention will become more apparent from the following detailed description of preferred embodiments of the invention and the accompanying drawing.

### BRIEF DESCRIPTION OF THE DRAWING

FIG. 1A is a graph showing changes in bi-fold skin thickness in response to various treatments (as measured).

FIG. 1B is a graph showing changes in bi-fold skin thickness in response to various treatments (in percent).

FIG. 1C is a graph showing changes in bi-fold skin thickness in response to various concentrations of EGCG.

FIG. 1D is a graph showing changes in bi-fold skin thickness in response to various concentrations of EGCG in combination with ascorbic acid.

FIG. 2 is a graph showing intracellular concentrations of hydrogen peroxide in response to various treatments.

FIG. 3A is a graph showing intracellular protein oxidation in response to various treatments.

FIG. 3B is a photograph of a western blot depicting intracellular protein oxidation in response to various treatments.

FIG. 4 is a is a photograph of a western blot depicting intracellular matrix metalloproteinase expression in response to various treatments.

### DETAILED DESCRIPTION

Solar ultraviolet (UV) radiation has been reported as the primary cause for the vast majority of cutaneous age-related diseases in human. Among other etiologic agents, various studies suggested that reactive oxygen species (ROS) may be involved in damage of critical cellular macromolecules (e.g., DNA, proteins, lipids), especially where the oxidative potential of the ROS exceeds cellular anti-oxidant potential.

Based on these and other observations, the inventors contemplate that oxidative stress may be associated with photoaging of the skin. Particularly, the inventors contemplate that oxidative stress may activate selected cellular signal transduction pathways, leading leading directly or indirectly to phosphorylation and induction of dermal matrix metalloproteinases (MMP). Among other causes, activation of MMPs degrade extracellular matrix proteins and may lead to wrinkling, photoaging, and/or other skin disorders. The inventors discovered that exposure of UV radiation to skin further exacerbates oxidative stress and phosphorylation of matrix metalloproteinases (MMPs), which is thought to play a crucial role in cutaneous photoaging.

Here, the inventors have unexpectedly discovered that ultraviolet (UV) light-induced oxidative stress and phosphorylation of MMPs can be prevented by synergistic combinations of a catechin (and particularly a mixture of catechins [e.g., polyphenon E and/or other green tea catechins]) with an antioxidant (especially ascorbic acid) at a relatively narrow range of synergistic combinations. Viewed from a different perspective, moderate protective effects can be observed for either compound (i.e., catechin or antioxidant alone) over a relatively wide range of concentrations. However, when combined to a specific combination (see data below), a substantial and synergistic protective effect against photoageing is achieved.

In one exemplary topical formulation, a combination of polyphenon E or EGCG and ascorbic acid was applied to skin in a hydrophilic cream to deposit the polyphenon E/EGCG and ascorbic acid in an amount of 2.0 mg/cm² and 1.0 mg/cm² to the skin, respectively, before single and multiple exposure to UV (90 mJ/cm²) for a period of one month. Remarkably, such treatment markedly decreased oxidative stress as measured by a reduction in the generation of hydrogen peroxide (56-73%) and nitric oxide (30-68%). In the same way, such treatment also significantly reduced epidermal lipid peroxidation (41-64%) and protein oxidation (56-90%). Furthermore, the observed molecular changes were also accompanied by a significant inhibition of UV-induced infiltration of CD11b+ cells, which are thought substantially contribute to the presence of reactive oxygen species in UV-irradiated skin. Additionally, the topical treatment also resulted in inhibition of UVB-induced phosphorylation of aging-related MMPs-2, -7 and -9 (41-66%).

In another exemplary treatment, mice were exposed to UVB (90 mJ/cm²) for a period of one month on alternate days with or without polyphenone E in the drinking water (0.2%, w/v). Remarkably, this protocol inhibited UV-induced markers of oxidative stress, however, to a lesser extent than topical treatment. The chemopreventive efficacy of EGCG was superior to polyphenone E. Further, skin appearance in polyphenone E or EGCG plus UV exposed mice was relatively better in comparison to UV alone exposed skin sites.

Based on the inventors' observations, it is contemplated that cutaneous photoageing can be reduced, or even prevented by administration of synergistic combinations of a catechin and an antioxidant, and particularly of a green tea catechin (e.g., EGCG or polyphenon E) and ascorbic acid. As a consequence of such treatment, it is further contemplated that prevention of oxidative stress and phosphorylation of MMPs may advantageously provide increased ease of wound healing, increased resiliency of skin towards mechanical injury, and an increase in resistance to infectious diseases of the skin.

With respect to suitable catechins, it should be appreciated that catechins isolated from plants are particularly preferred, and especially suitable catechins include those isolated from green tea. Therefore, contemplated catechins especially include (−)-epigallocatechin gallate, (−)-epigallocatechin, (−)-gallocatechin gallate, (−)-gallocatechin, (−)-epicatechin gallate, (−)-epicatechin, (−)-catechin gallate, and (+)-catechin. Alternative sources for catechins include black tea, oolong tea, apple, pears, and wine (and other fermented and unfermented grape extracts), etc. Moreover, the catechin may be included in contemplated formulations as a single compound, or as one of a plurality of chemically distinct catechins. Typically, where more than one catechin is used in topical formulations, the catechins are provided in form of a plant extract, and most typically as a green tea extract. Among other suitable extracts, especially contemplated catechin preparations include commercially available polyphenon E and polyphenon B.

Where desired, the catechin may be chemically modified to improve at least one of chemical stability (and especially oxidation), render the catechin more lipophilic, and/or to add one or more physiologically desirable properties. There are numerous methods for chemical modification of catechins known in the art, and all of these are deemed suitable for use herein. Exemplary modifications are described in U.S. Pat. App. No. US20050014958, U.S. Pat. No. 6,562,864, or in Japanese patent application with the publication number JP57120584. Still further, while catechins used in conjunction with the inventive subject matter presented herein are preferably isolated from a natural source, synthetic catechins are also deemed suitable.

With respect to the antioxidant, it should be appreciated that all known antioxidants are contemplated herein. However, particularly preferred antioxidants are pharmaceutically acceptable antioxidants. Moreover, and especially where the topical formulation is a hydrophilic preparation, it is preferred that the antioxidant is a hydrophilic (e.g., at least 10 mg per ml) antioxidant. For example, suitable hydrophilic antioxidants include ascorbic acid, carnosine, dimethylthiourea, and chemical derivatives (various esters, and amides) thereof. Alternatively, the hydrophilic antioxidant may also be provided in form of an antioxidative extract or solution, and particularly preferred solutions and extracts include fruit extracts enriched in ascorbate.

On the other hand, in less preferred aspects of the inventive subject matter, it is also contemplated that at least a portion of the total antioxidants may be provided as hydrophobic antioxidant. Consequently, suitable hydrophobic antioxidants also include various optionally substituted tocopherols, lycopenes, and carotenes. While not limiting to the inventive concept presented herein, it is contemplated that the antioxidant will have at least a two-fold effect in contemplated topical compositions. For example, the antioxidant may help prevent oxidation of the catechin as well as reduce overall oxidative stress in the skin. Similarly, the catechin may exert desirable physiological effects in more than one manner. For example, the catechin may act as an antioxidant and as a modulator of inflammation or stress response pathways. Thus, synergistic action may be achieved by a combination of desirable effects that enhance each other at suitable concentrations.

In most preferred aspects, the catechin is present at about a two-fold excess over the antioxidant (on a weight/weight basis). Viewed from another perspective, it is generally preferred that the catechin is present in an about five-fold molar excess (based on EGCG). Therefore, especially contemplated topical formulations will include the catechin and the hydrophilic antioxidant at a ratio of between 2.7 to 1.2 (by weight), more preferably between 2.5 to 1.5 (by weight), even more preferably between 2.3 to 1.7 (by weight), and most preferably between about 2.1 to 1.9 (by weight). Similarly, the catechin in contemplated formulations will be present in a molar excess of between 3-fold to 8-fold, more preferably between 4-fold to 7-fold, and most preferably between 5-fold and 6-fold.

Moreover, it is preferred that the catechin and the (typically hydrophilic) antioxidant are present in the formulation at synergistic concentrations to achieve a reduction of photoageing. For example, and among other things, suitable markers for such reduction include a reduction of UV-induced skin thickness, a reduction of hydrogen peroxide radical formation in skin, a reduction of protein oxidation in skin, and a reduction of expression of an MMP (i.e., matrix metalloproteinase). In most preferred aspects, the catechin and (typically hydrophilic) antioxidant are present in the formulation in an amount such that application of the composition will deposit the catechin at an amount of between 0.35 mg/cm² and 1.7 mg/cm², more preferably between 0.5 mg/cm² and 1.5 mg/cm², even more preferably between 0.7 mg/cm² and 1.3 mg/cm², and most preferably between 0.85 mg/cm² and 1.15 mg/cm².

Viewed from a different perspective, the catechin and the antioxidant will be present in an amount effective to reduce UV-induced skin thickness in an amount of at least 60%, and more preferably at least 75% as compared to non-treatment. Additionally, or alternatively, the catechin and the antioxidant will be present in an amount effective to reduce hydrogen peroxide radical formation in an amount of at least 40%, more typically at least 45%, and most typically at least 50% as compared to non-treatment. Similarly, it is contemplated that the catechin and the antioxidant will be present in an amount effective to reduce protein oxidation in skin in an amount of at least 60%, and more typically at least 70%, and/or to reduce expression of a matrix metalloproteinase in an amount of at least 20%, and more typically at least 30%.

Additionally, it should be recognized that contemplated topical formulations may also other active ingredients, including UV-absorbing compounds, moisturizing compounds, alpha hydroxy acids, and compounds that promote collagen synthesis. Particularly preferred compounds that absorb UV are those with a molar extinction coefficient of at least 1000 cm⁻¹ at a wavelength of between 290 nm and 390 nm. For example, suitable compounds include 3-imidazol-4-yl acrylate, salicylate, p-methoxy cinnamate, 2-ethyl-hexyl-2-cyano-3,3-diphenyl acrylate, 3,3,5-trimethylcyclohexyl-2-acetamido benzoate, p-aminobenzoate, cinnamate, 3,4-dimethoxy phenyl glyoxylate, α-(2-oxoborn-3-ylidene)-p-xylene-2-sulphonate, α-(2-oxoborn-3-ylidene) toluene-4-sulphonate, α-cyano-4-methoxy cinnamate, 2-phenyl-benzimidazole-5-sulphonate, 2-hydroxy-4-methoxy benzophenone-5-sulphonate, 2,2′-dihydroxy-4,4′-dimethoxy-benzophenone-3,3′-disulphonate. Suitable moisturizing compounds include ceramides, various polyols (e.g., propylene glycol, glycerine, sorbitol, hyaluronic acid), collagen, etc., while suitable alpha hydroxy acids include lactic acid, glycolic acid, malic acid, citric acid, etc., and suitable collagen synthesis promoters include GHK-Cu²⁺ complexes.

It should be recognized that the catechin and antioxidant may be formulated in numerous topical formulations, and especially preferred formulations include hydrophilic topical preparation well known in the art (e.g., cream, mousse, lotion, or spray). For example, suitable topical formulations are described in “Cosmetic and Toiletry Formulations”, Volume 8, by Ernest Flick (Noyes Publications; 2nd edition (Jan. 15, 2000); ISBN: 0815514549), which is incorporated by reference herein.

Further contemplated topical formulations preferably include hydrophilic, aqueous mixtures such as a solution, colloidal solution, emulsified lotion, O/W cream (hydrophilic cream) and aqueous gel wherein the aqueous phase is the continuous phase. Alternatively, contemplated hydrophobic oily mixtures such as oil solutions, ointments, hydrophobic gels (e.g., mineral oil gelled with polyethylene) are also deemed suitable in which an emulsifier is added to the oil (here, the oil phase is the continuous phase).

Hydrophilic components typically include aqueous solutions, which may further include hydrophilic components (e.g., glycerol, carbohydrates, etc.), while hydrophobic components include hydrocarbons (e.g., liquid paraffin, vaseline, solid paraffin, microcrystalline wax, etc.). Emulsifiers and dispersing agents may be included and exemplary compounds are anionic, cationic and nonionic surfactants. Nonionic surfactants are preferred because of their low levels of irritation to skin. Typical of nonionic surfactants are fatty acid monoglycerides, sorbitan fatty acid esters, sucrose fatty acid esters, polyoxyethylene fatty acid esters, and polyoxyethylene higher alcohol ethers. Still further, gelatinizers may be employed where desirable and especially include carboxymethylcellulose, cellulose gel, carbopol, polyvinyl alcohol, polyethylene glycol and various gums.

In order to further increase the stability of the topical preparation, chelating agents (e.g., EDTA, thioglycolic acid, thiolactic acid, thioglycerine), antiseptics (e.g., methyl, ethyl, propyl and butyl esters of p-hydroxybenzoic acid, o-phenylphenol, dehydroacetic acid), or other preservatives may be added. It is still further preferred that the pH is adjusted to a neutral or even slightly acid pH to match or approximate the pH of healthy skin. Suitable acidifiers especially include citric acid, lactic acid, tartaric acid or the like.

### EXPERIMENTS

### Animal Studies

SKH-1 hairless mice were used at an age of between 6-8 weeks. The mice were UVB exposed (90 mJ/cm²) for two months on alternate days for the present photoageing model. At the termination of the experiment, mice were sacrificed 24 hr after the last UV exposure, skin biopsies were collected for analyzing various parameters.

Ascorbic acid and EGCG (or polyphenon E) were dissolved at various concentrations in a hydrophilic cream and were topically applied on the mouse skin 25-30 min before each exposure of UVB. In preliminary studies, we tested the efficacy of AA and EGCG dose-dependently against UVB-induced adverse effects in the skin. We found that the application of 1 mg EGCG/cm² skin area resulted in significant chemopreventive effects against UVB radiation. Therefore, to evaluate the anti-photoaging effects, we used this dose in all the experiments performed.

### Biomarkers for Evaluation of Anti-Photoaging Effect of Ascorbic Acid and EGCG

1. Bi-fold skin thickness of the UV exposed skin site with or without the treatment of ascorbic acid (AA) and EGCG.

2. Hydrogen peroxide production as a marker of oxidative stress.

3. Protein oxidation.

4. Matrix metalloproteinases, like MMP-2, MMP-3, MMP-7 and MMP-9 which play a major role in degradation of extracellular matrix of the skin and leads to skin aging or wrinkle formation.

5. Tissue inhibitor of matrix metalloproteinase (TIMP). The induction of TIMP with AA or EGCG treatment may be involved in the inhibition of MMP expression.

### Experimental Results

Topical treatment with AA, EGCG, and combinations of AA and EGCG resulted in varying degrees of protection against UVB-induced damage to skin. Control experiments were performed to verify skin damage due to UVB exposure at the above dosage regimen. Typically, UVB-damaged skin had a rough skin appearance, and skin thickness was increased as determined by bi-fold skin thickness. In the below experiments, mice were irradiated to UVB (90 mJ/cm²) for two months on alternate days to effect photoageing of the skin. Mice were topically treated either with AA, EGCG or a combination of AA (unless indicated otherwise at 0.5 mg EGCG/cm² skin area) and EGCG (unless indicated otherwise at 1 mg EGCG/cm² skin area) before each exposure of the UVB to determine the photoprotective effect of these agents. Hydrophilic cream was used as a vehicle. UVB alone irradiated mice (control) were topically treated with vehicle only before UVB exposure.

Based on various experiments (data not shown), the optimum dosage for ascorbic acid was determined to be about 0.5 mg/cm² of skin. In most cases, dosages of less than 0.5 mg/cm² resulted in a decreased protection (as measured by bi-fold skin thickness), while doses substantially above 0.5 mg/cm² of skin tended to provoke an inflammatory reaction. Therefore, and based on these findings, dosages of ascorbic acid for selected experiments were maintained at about or below 0.5 mg/cm².

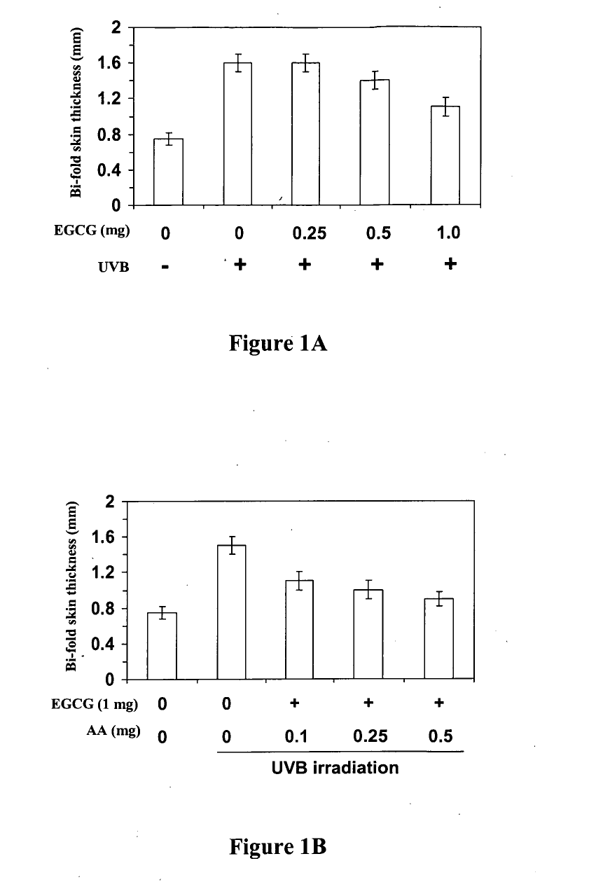

FIG. 1A depicts a graph in which topical dosage of EGCG was correlated with a photoprotective effect as measured by the bi-fold thickness test. Here, the photoprotection increased to a dosage of about 1.0 mg/cm² of skin, while providing little or no further beneficial effect at increasing dosages. FIG. 1B depicts a graph in which varying dosages of ascorbic acid (between 0.1 and 0.5 mg/cm²) were evaluated in combination with a fixed dosage of EGCG (1 mg/cm²). Remarkably, almost complete photoprotection was achieved at a treatment where EGCG was present at a dosage of about 1 mg/cm² and ascorbic acid at a dosage of about 0.5 mg/cm² (observed effect between untreated control and synergistic combination was in some cases within margins of error).

To determine if the effect was additive or synergistic, ascorbic acid and EGCG were topically used at the above determined optimum dosages. As depicted in FIGS. 1C and 1D, topical treatment using combinations of AA and EGCG resulted in significant prevention of UVB-induced skin damage as measured by the bi-fold thickness test, whereas individual treatments provided substantially less protection. It was also observed that subjective skin appearance was much better than non-AA and non-EGCG treated skin sites. Interestingly, the combined effect of AA and EGCG was greater than individual effect of AA or EGCG. Data in parentheses of FIG. 1D indicate the percent protection against UVB-induced increase in bi-fold skin thickness.

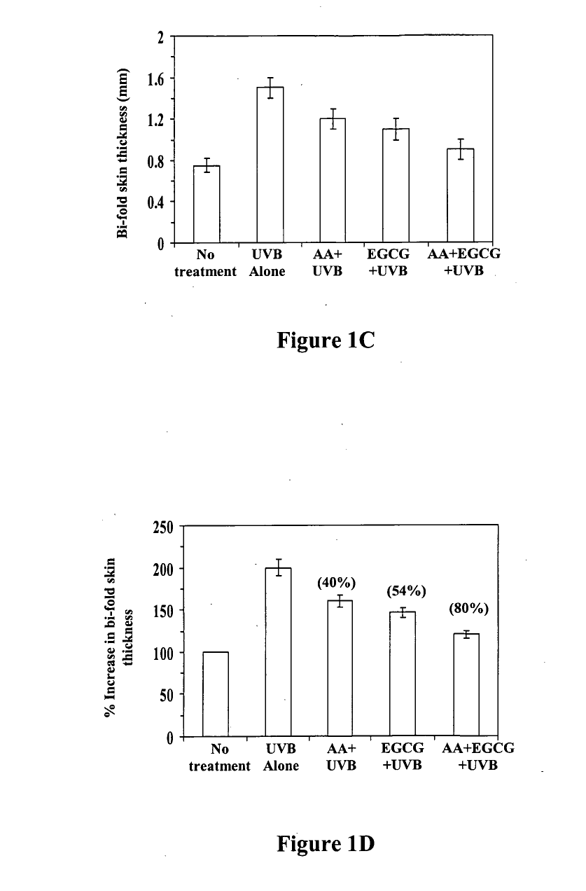

To further identify beneficial effects of the synergistic combination of ascorbic acid and EGCG (EGCG dosage of about 1 mg/cm² and ascorbic acid dosage of about 0.5 mg/cm²), intracellular release of H₂O₂ was measured as a marker of oxidative stress. After UVB exposure, a single cell suspension from the epidermis and dermis was prepared following procedures well known in the art. H₂O₂ was assayed using dihydrorhodamine 123 as a fluorescent dye probe. As evidenced by the control experiments and depicted in FIG. 2, topical treatment with ascorbic acid and EGCG individually did not induce H₂O₂ production in skin cells, while UVB irradiation of the skin resulted in significant formation of H₂O₂. Treatment with ascorbic acid and EGCG inhibited UVB-induced H₂O₂ production in cells. Again, the observed combined effect of ascorbic acid and EGCG was greater than individual inhibitory effects of ascorbic acid and EGCG.

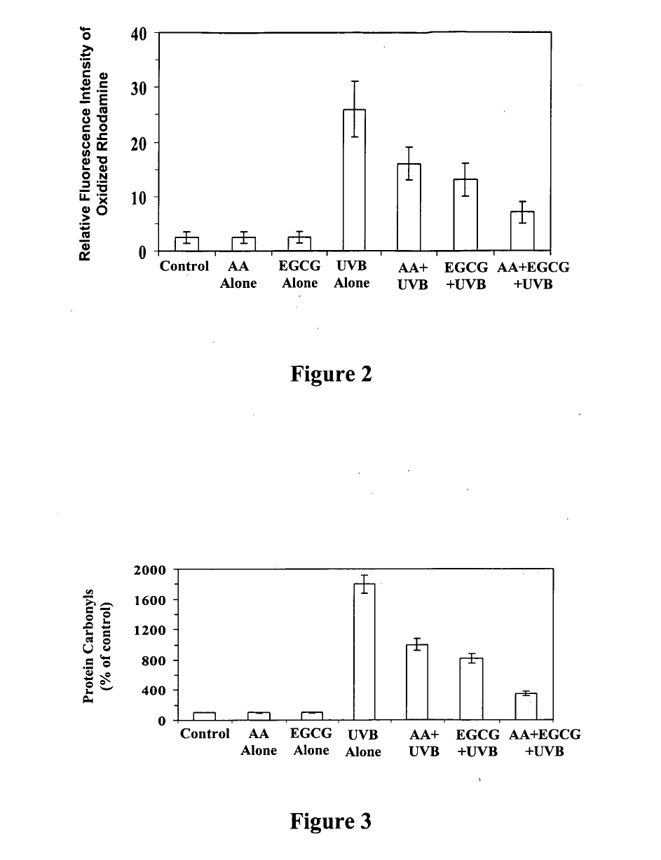

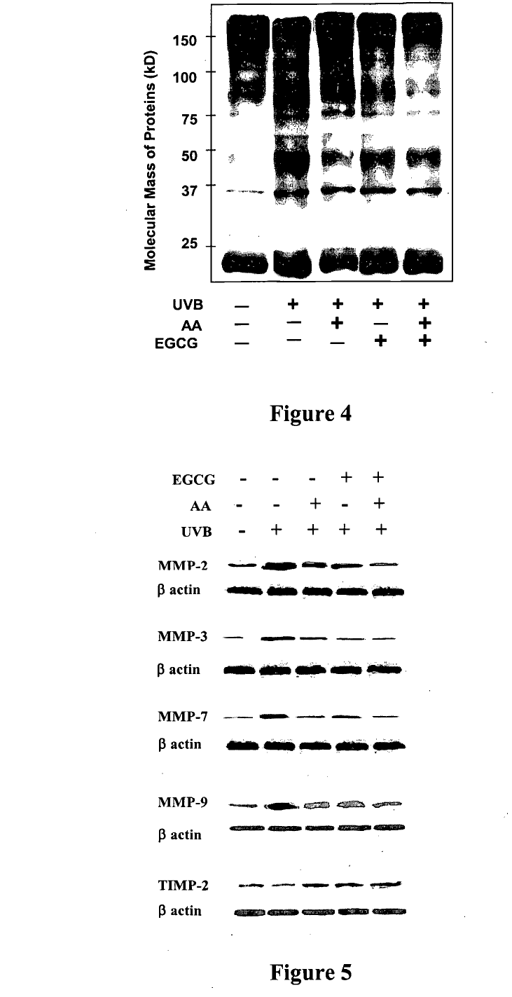

In a further series of experiments, UVB-induced oxidation of proteins in the skin was measured and plotted as a function of topical treatment with ascorbic acid, EGCG, and the synergistic combination of ascorbic acid and EGCG (EGCG dosage of about 1 mg/cm² and ascorbic acid dosage of about 0.5 mg/cm²) as oxidation of proteins has been associated with photodamage of skin. As shown in FIG. 3, protein oxidation was determined in terms of protein carbonyl formation by routine analytical methods. FIG. 4 depicts a western blot analysis using antibodies against carbonyl-containing proteins. Protein oxidation was determined following western blot analysis by using OxyBlot Protein Oxidation Detection kit (Intergen Company, Purchase, N.Y.). Both ascorbic acid and EGCG individually inhibited UVB-induced oxidation of proteins to some degree. However, when ascorbic acid and EGCG were combined in the synergistic combination, the effect of ascorbic acid and EGCG was greater than individual effects.

The effects of topical treatments with ascorbic acid, EGCG, and the synergistic combination of ascorbic acid and EGCG (EGCG dosage of about 1 mg/cm² and ascorbic acid dosage of about 0.5 mg/cm²) were also determined on UVB-induced expression of selected matrix metalloproteinases (MMP) as it was previously shown that activation or expression of MMP is associated with degradation of extracellular matrix proteins. Most matrix proteins provide tensile strength to the skin, and are therefore thought to be associated with photoageing and/or wrinkling of skin. We previously reported that chronic exposure of skin to UVB induces the up-regulation of several MMP, particularly, MMP-2, MMP-3, MMP-7 and MMP-9. As shown in FIG. 5, topical treatment with ascorbic acid and EGCG individually inhibited UVB-induced expression of selected MMP to at least some degree as determined by western blot. The blots were stripped and re-probed for β-actin antibody to verify equal protein loading and equal transfer of proteins from gel to membrane. Clearly, the combined effect of ascorbic acid and EGCG was again substantially higher than the individual effects. Moreover, ascorbic acid and EGCG also up-regulated the expression of TIMP which might be responsible for the inhibition of activation of MMP in UVB exposed skin.

### Substitution of Topical EGCG with Oral Polyphenon E

To investigate an alternative route of administration of at least one of the components in contemplated agents against photoageing, the inventors replaced topical administration of EGCG with oral administration of EGCG. Mice were exposed to a single UV exposure of 180 mJ/cm², animals were sacrificed 24 h after UV exposure. Skin biopsies were collected from the mice of each group. Single cell suspension was prepared and subjected to determination of H₂O₂ production using dihydrorhodamine 123 (DHR) as a fluorescent dye probe. In multiple UV exposure, mice were exposed to 90 mJ/cm² for one month on alternate days, and mice were sacrificed 24 h after the last exposure of UV. Lipid peroxidation was determined in microsomal fraction of the skin samples (MDA is malondialdehyde). The data in parentheses in the table below indicate the percent inhibition by Polyphenon E treatment in drinking water.

| | | |
|---|---|---|
| | H₂O₂ Production | |
| | Relative fluorescence of | Lipid Peroxidation |
| Treatment Groups | Rhodamine 123 | nmole MDA/mg protein |

Single UV exposure

| | | |
|---|---|---|
| Control | 4 ± 2 | 0.14 ± 0.01 |
| UV exposed | 22 ± 6 | 0.43 ± 0.02 |
| Polyphenon E + UV | 16 ± 6 (33%) | 0.31 ± 0.02 (41%) |

Multiple UV exposure

| | | |
|---|---|---|
| Control | 5 ± 02 | 0.14 ± 0.01 |
| UV exposed | 31 ± 11 | 0.71 ± 0.06 |
| Polyphenon E + UV | 21 ± 90 (39%) | 0.46 ± 0.02 (44%) |

Remarkably, the inventors discovered that topical EGCG administration for UVB photoprotection can be replaced with oral administration, where either EGCG is administered alone (data not shown) or in combination with other catechins (here: polyphenon E).

### Exemplary Hydrophilic Cream

| INGREDIENT | WEIGHT PERCENT |
|---|---|
| Glyceryl cocoate | 34.0 |
| Glyceryl trilaurate | 5.0 |
| Glycerin | 13.0 |
| EDTA | 0.2 |
| Phosphate buffer (pH 7.0) | 43.3 |
| PolyphenonE | 3.0 |
| Ascorbic acid | 1.5 |

Glyceryl cocoate, glyceryl trilaurate and glycerin will be mixed together and heated to 60° C. In a separate container, the EDTA, ascorbate, and phosphate buffer (0.3M Na2 HPO4, pH 7.0) will be combined and heated to 60° C. The buffer solution will then be added to the glyceryl-containing solution and cooled with mixing to 40° C. The polyphenonE, will then be slowly added with mixing and allowed to cool to room temperature.

Thus, specific embodiments and applications for reduction of photoageing have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. A topical hydrophilic composition for reducing photoageing comprising a catechin and a hydrophilic antioxidant at a ratio of between 2.3 to 1.7 (by weight) and present in an amount such that application of the composition will deposit the catechin at a dosage of between 0.7 mg/cm² and 1.3 mg/cm².

2. The composition of claim 1 wherein the catechin is epigallocatechin gallate.

3. The composition of claim 1 wherein the catechin is provided with a plurality of additional catechins.

4. The composition of claim 3 wherein the catechin and the plurality of additional catechins is polyphenon E.

5. The composition of claim 1 wherein the hydrophilic antioxidant is ascorbic acid or a substituted ascorbic acid.

6. The composition of claim 1 further comprising a compound that absorbs UV with a molar extinction coefficient of at least 1000 cm⁻¹ at a wavelength of between 290 nm to 390 nm.

7. The composition of claim 1 wherein the ratio between the catechin and the antioxidant is between 2.1 to 1.9.

8. The composition of claim 1 wherein reduction of photoageing is characterized by at least one of a reduction of UV-induced skin thickness, a reduction of hydrogen peroxide radical formation in skin, a reduction of protein oxidation in skin, and a reduction of expression of a matrix metalloproteinase.

9. The composition of claim 8 wherein the reduction of UV-induced skin thickness is at least 75% as compared to non-treatment.

10. The composition of claim 8 wherein the reduction of hydrogen peroxide radical formation is at least 50% as compared to non-treatment.

11. A method of reducing photoageing comprising:
providing a catechin and a hydrophilic antioxidant in a'hydrophilic composition at a ratio of between 2.3 to 1.7 (by weight);
depositing the composition on skin in an amount such that the catechin is present on the skin at a dosage of between 0.7 mg/cm² and 1.3 mg/cm²; and
irradiating the skin with UV-B radiation.

12. The method of claim 11 wherein the catechin is epigallocatechin gallate.

13. The composition of claim 11 wherein the catechin is provided with a plurality of additional catechins.

14. The composition of claim 13 wherein the catechin and the plurality of additional catechins is polyphenon E.

15. The composition of claim 11 wherein the hydrophilic antioxidant is ascorbic acid or a substituted ascorbic acid.

16. The composition of claim 11 further comprising a compound that absorbs UV with a molar extinction coefficient of at least 1000 cm⁻¹ at a wavelength of between 290 nm to 390 nm.

17. The composition of claim 11 wherein the ratio between the catechin and the antioxidant is between 2.1 to 1.9.

18. The composition of claim 11 wherein reduction of photoageing is characterized by at least one of a reduction of UV-induced skin thickness, a reduction of hydrogen peroxide radical formation in skin, a reduction of protein oxidation in skin, and a reduction of expression of a matrix metalloproteinase.

19. The composition of claim 18 wherein the reduction of UV-induced skin thickness is at least 75% as compared to non-treatment.

20. The composition of claim 18 wherein the reduction of hydrogen peroxide radical formation is at least 50% as compared to non-treatment.
